(19) United States
(12) Patent Application Publication
(10) Pub. No.: US 2012/0202292 A1
(43) Pub. Date: Aug. 9, 2012

(54) Novel method to generate nutritional compounds from microalgae

(75) Inventors: Chifu Huang, Lexington, KY (US); Jeffrey Lee Ebersole, Lexington, KY (US)

(73) Assignee: Chifu Huang, Lexington, KY (US)

(21) Appl. No.: 13/021,764

(22) Filed: Feb. 6, 2011

(51) Int. Cl. C12N 15/79

(52) U.S. Cl. 435/471

(57) ABSTRACT

The present invention relates to the application of genomics technology to generate nutritional compounds in algae, such as microalgae. The present invention discloses a novel method to modify algae to produce a variety of nutritional compounds. Specifically, this method utilizes microalgae genomics technology to manipulate the existing metabolic pathways in the genomes of microalgae and therefore induce the algae to modify secondary metabolic pathways to increase the level of existing nutritional compounds or produce novel nutritional compounds with a variety of commercial applications for improving human and animal nutrition and health.

### FIELD OF THE INVENTION

Algae-based natural products can be used as nutritional dietary supplements or as food sources. For example, the blue-green alga Spirulina can be used for human and animal food; and the pigment of red algae is widely used as a dietary supplement. However, microalgae have not been previously reported to be used as a source of food and nutrition. Even though microalgae possess a broad metabolic capacity, the algae do not always reveal their functional potential due to various environmental constraints and produce limited nutritional compounds that would be directly needed for their survival and growth under the specific environment conditions. Genomics technology has been successfully used in plant and agricultural crops to produce novel traits in plants, such as disease-resistance or insect-resistance properties. However, genomics technology has seldom been applied to algae. In this invention, we have developed a novel technology, called Microalgae Genomics Technology™ (MaGT), which could produce recombinant algae that possess new traits and produce novel nutritional compounds or produce higher levels of existing nutritional compounds with commercial application. This method utilizes microalgae genomics technology to manipulate the metabolic pathways in the genomes of microalgae and therefore induce the algae to modify secondary metabolic pathway to increase the concentration of existing nutritional compounds or novel compounds with a variety of commercial applications and nutritional values for human and animals.

### BACKGROUND OF THE INVENTION

Microalgae can be grown in mass in both open-culture systems such as ponds, lakes and raceways, or in highly controlled closed-culture systems, similar to those used in commercial fermentation processes, such as for E. coli and mammalian cells. Certain microalgae are very suitable for open system culture where the environmental conditions are very specific, such as high salt or high alkaline ponds, lakes, or lagoons. The extreme nature of these environments severely limits the growth of competitive species, although other types of organisms may still contaminate the culture. The advantage of such systems is that they are generally a low investment, very cost-effective, with high added value, and easy to manage. Closed-culture systems, on the other hand, require significantly higher investments and operating costs, but are independent of all variations in agro-climatic conditions and are very closely controlled for optimal performance and quality. Algae products have substantial potential to be explored for food, medicinal, or energy uses by man. The repertoire of algae products can be explored in the future to meet human's increasing demand for consumption for various products. It is reported that the algae genome shows approximately 120 million nucleotides, and shares nearly 7,000 genes with other organisms, with more than a third of these being shared with both humans and flowering plants.

Currently, most specific algae of commercial value are developed under certain nutritional or environmental selection. There is no genomic method available to stimulate or make these algae species produce novel nutritional compounds. There is a strong need in algae for such a technology and market potential for the algal nutritional compounds, which is cost-effect and nutrition enriched. A proprietary platform biotechnology, Microalgae Genomics Technology™ (MaGT), has been developed by us. The basis of MaGT™ is that random activation mutations are induced in a large number of microalgal cells. With this MaGT technology (see examples) and HTS screening method, we were able to produce nutritional compounds in the mutant algae which exhibited the desired property.

### SUMMARY OF THE INVENTION

The present invention is to utilize a novel method, Microalgae Genomics Technology™ (MaGT), in microalgae to produce novel nutritional compounds or increase the yield of existing nutritional compounds. Specifically, the Microalgae Genomics Technology™ (MaGT) utilizes random activation mutations, which are introduced into a large number of microalgae cells. These cells then form a population of mutant clones, in which each clone is over-expressing one or more activated genes. Some of these mutants may have activated genes involved in the enzymes of secondary metabolic pathways. These algae mutants may produce novel nutritional compounds or increase the yield for existing nutritional compounds. The Microalgae Genomics Technology™ (MaGT) is invented as a novel method and a new art to generate chemicals of nutritional use, but not limited to.

The characteristics of the above technology are:

(1) The Microalgae Genomics Technology™ (MaGT) utilizes random activation mutations through agrobacteria, which induce in a large number of mutant microalgal cells. These cells then form a population of mutant clones, in which each clone is over-expressing one or more activated genes. Some of these mutants may have activated genes involved in the enzyme pathways of secondary metabolites.
(2) MaGT can be used to generate novel bioactive compounds. For example, the novel compounds with nutritional property could be generated and high-throughput screening method is used to screen for the nutritional property.
(3) MaGT can also be used to generate novel nutritional compounds or to increase the amount of nutritional compounds in microalgae, but not limited to microalgae. It can also be used in other algae species, such as macroalgae.
(4) MaGT can be used to generate a profile of nutritional compounds.

### DETAILED DESCRIPTION OF THE INVENTION

The following examples are presented by way of illustration, not of limitation.

### Example 1

First, the microalgae species, Chlorella vulgaris, are mutagenized using activation tagging mutagenesis (ATM) by transfer of DNA from Agrobacterium tumefaciens into the algae genome. This randomly incorporates viral enhancer sequences together with a bacterial gene for antibiotic resistance into the microalgae genome. The algae cells are then selected for the ability to grow in hygromycin-containing medium, which indicates that the “tag” (T-DNA) construct has been successfully incorporated. The enhancer sequences activate the expression of genes in the immediate vicinity of their site of incorporation and, in the case of C. vulgaris, a population of mutant cells should be obtained with the genome containing activation mutations. These cells were then allowed to grow to form recombinant clones in semi-solidified medium. These clones are screened and isolated by picking individual clones and testing for their nutritional properties. In some cultures, the random activation of a gene or genes by ATM should have a specific impact on secondary metabolism of those clonal cultures. This process is the key component of our Microalgae Genomics Technology™ (MaGT)

### Example 2

For the protein nutrient, the protein concentration of the recombinant algae clones was analyzed using a BioRad® assay. Recombinant C. vulgaris clones were isolated and cultured in 96-well plates. Extracts were prepared from each clone and were tested for protein concentration using the above assay. Three recombinant Chlorella algae clones with the highest protein levels were identified, as compared to control culture extracts.

### Example 3

For the carbohydrate nutrient, the carbohydrate concentration of the recombinant algae clones was analyzed with a phenol-sulfuric acid assay, which detects the carbohydrate concentration. Recombinant C. vulgaris clones were isolated and cultured in 96-well plates. Extracts were prepared from each clone and were tested for carbohydrate concentration with phenol-sulfuric acid assay. Two clones were identified which showed strong antibacterial activity, as compared to control culture extracts.

### Example 4

For the lipid nutrient, the lipid concentration of the recombinant algae clones was analyzed with GC/MS analysis and thin layer chromatography (TLC). For example, the lipid concentration of the recombinant algae clones was analyzed with a GC/MS analysis. Recombinant C. vulgaris clones were isolated and cultured in 96-well plates. Extracts were prepared from each clone and were esterised with ethanol. The esters were tested for lipid concentration by GC/MS analysis. Three clones were identified which showed the highest lipid concentration.

### Example 5

For example, the fatty acid concentration of the recombinant algae clones was analyzed with a GC/MS analysis and thin layer chromatography (TLC). The fatty acids could be eicosapentaenoic acid (EPA), docosahexaenoic Acid (DHA), or α-linolenic acid (ALA). Recombinant C. vulgaris clones were isolated and cultured in 96-well plates. Extracts were prepared from each clone and were tested for fatty acids concentration. Three clones were identified which showed the highest EPA concentration.

### Example 6

A HTS (high-throughput screening) method was used to screen for nutritional compounds. For the phytochemicals, for example of polyphenols, the polyphenol concentration of the recombinant algae clones was subjected to an HPLC analysis. Recombinant C. vulgaris clones were isolated and cultured in 96-well plates. Extract were prepared from each clone and were tested for polyphenol concentration by HPLC. Four clones were identified, which showed the highest polyphenol concentration.

### BEST MODE FOR CARRYING OUT THE INVENTION

The preferred embodiments of this invention are described herein, which includes the best mode that is known to the inventor for implementing the invention. Even though the invention has been described with reference to particular embodiments, it is to be understood that these embodiments are merely illustrative of the applications and principles of the invention. Therefore, it is to be understood that many modifications may be made to the illustrative embodiments without departing from the spirit and the scope of the invention as defined in the attached claims.

### INDUSTRIAL APPLICABILITY

This invention technology is capable of producing novel nutritional compounds in algae and microalgae in large scale, and can be used in a variety of applications where the nutritional value of algae can be increased or enriched. These novel nutritional compounds could be used as ingredients for consumer goods including foods and beverages, medical devices, medical supplies, dental products, pharmaceuticals, functional foods, drugs, and/or in industrial products.

In addition, this invention technology is also capable of increasing the yields of existing nutritional compounds in algae and microalgae. These existing nutritional compounds could be used as ingredients for consumer goods including foods and beverages, medical devices, medical supplies, dental products, pharmaceuticals, functional foods, drugs, and/or in industrial products.

1. A microalgae-based method, called Microalgae Genomics Technology™ (MaGT), which can manipulate the metabolic pathways in the genome of microalgae, such as Chlorella vulgaris, to generate novel nutrients or increase existing nutrients, which could have potential commercial use for humans and animals as food and dietary supplements, but not limited to.

2. A microalgae-based method, called Microalgae Genomics Technology™ (MaGT), which can manipulate the metabolic pathways in the genome of microalgae, such as Chlorella vulgaris, to generate nutritional compounds; but not limited to Chlorella algae, it can apply to other microalgae, such as Spirulina algae, Scenedesmus algae, Cyanobacteria, Haematococcus, Crypthecodinium, Schizochytrium, Dunaliella, Odontella, Porphyridium, Shizochytrium, Isochrysis. For example, Spirulina platensis, Dunaliella salina, Haematococcus pluvialis, Porphyridium cruentum, Scenedesmus almeriensis, Phaeodactylum tricornutum, Dunaliella bardawil, Chlamydomonas nivalis, Porphyridium cruentum, Phaeodactylum tricornutum, Scenedesmus obliquus, Isochrysis galbana, Porphyridium cruentum, Chlorella minutissima, Tetraselmis suecica, Tetraselmis suecica, Nannochloropsis, Isochrysis galbana, Scenedesmus almeriensis, Botryococcus braunii, Arthrospira maxima, Isochrysis galbana, Spirulina maxima, Diacronema vlkianum, Chaetoceros calcitrans, C. gracilis, S. costatum, T. pseudonana, Platymonas lutheri, Prymnesiophytes lutheri, Isochrysis sp. and Chroomonas salinaium.

3. A microalgae-based method, called Microalgae Genomics Technology™ (MaGT), which can manipulate the metabolic pathways in the genome of microalgae to generate nutritional compounds; but not limited to microalgae, it can apply to other algae, such as macroalgae as well, such as Halimeda, Caulerpa, Seaweed, Caulerpa, Fucus, Gracilaria, Laminaria, Macrocystis, Monostroma, Porphyra.

4. As mentioned in claim 1, the application of the Microalgae Genomics Technology™ (MaGT), which can manipulate the microalgae genome to generate novel nutritional compounds in microalgae, which could be used for human and animal as food and dietary supplements.

5. As mentioned in claim 1, the application of the Microalgae Genomics Technology™ (MaGT), which can manipulate the microalgae genome in algae to increase the levels of existing nutritional compounds in algae, which could be used for human and animals as food and dietary supplements.

6. As mentioned in claim 1, the application of the Microalgae Genomics Technology™ (MaGT), which can manipulate the microalgae genome to generate a profile of nutritional compounds in algae, which could be used for humans and animals, as food and dietary supplements, to improve overall nutritional health; or use a mixture of different algae with different nutritional values.

7. The application of the Microalgae Genomics Technology™ (MaGT), which can manipulate the microalgae genome to generate nutritional compounds, which include proteins, carbohydrates, fatty acids, minerals, amino acids, vitamins, sterols, but not limited to.

8. The application of the Microalgae Genomics Technology™ (MaGT), which can manipulate the microalgae genome to generate nutritional compounds, which include the algae secondary metabolites, such as phytochemicals, phytopigments, polyphenols, polysaccharides, dietary fibers, and flavonoids, but not limited to.
